SEQUENCE LISTING (2 sequences)

SEQ ID NO: 1 - protein, 120 aa, Macaca fascicularis
QVQLLESGGGLAKPGGSLRLSCAASGFTFTDYYMDWVRQAPGKGLEWVSRISPGGDVTWYADSVKGRFTISRDNAQNTLYLQMNSLRAEDTAVYFCARDDIVVSRIFDDWGQGVLLTVSS

SEQ ID NO: 2 - protein, 107 aa, Macaca fascicularis
ELQMTQSPSSLSASVGDRVTITCRASQSIRSYLAWYQQKPGKAPKLLIYDAAHLQSGVPSRFSGSGSGTEFSLTISSLQPEDFAVYYCQQRNSYPLTFGGGTKVEIK

(19) United States
(12) Patent Application Publication
(10) Pub. No.: US 2013/0184440 A1
(43) Pub. Date: Jul. 18, 2013

(54) SUPER-HUMANIZED ANTIBODIES

(71) Applicant: Philippe Thullier, La Tronche Cedex (FR)

(72) Inventor: Philippe Thullier, La Tronche Cedex (FR)

(73) Assignee: ETAT FRANÇAIS (MINISTÈRE DE LA DÉFENSE), SERVICE DE SANTÉ DES ARMÉES, Armees (FR)

(21) Appl. No.: 13/824,725

(22) PCT Filed: Sep. 21, 2010
(86) PCT No.: PCT/FR2010/051966

§ 371 Date: Mar. 18, 2013

(51) Int. Cl. C07K 16/00

(52) U.S. Cl. CPC C07K 16/00
USPC 530/387.3; 536/23.53; 435/320.1; 435/69.6

(57) ABSTRACT

A method of preparing a germinalized hypervariable antibody region directed against a target, as well as the antibodies, antibody fragments, vectors and compositions including the germinalized hypervariable region.

The present invention relates to a method of preparing hyperhumanized antibodies.

The monoclonal antibodies represent a therapeutic class that is in constant development. The recent, considerable advances of recombinant monoclonal antibodies (Abs) are notably due to the good tolerance of these molecules, which have developed from murine Abs to human Abs, passing through the intermediate generations consisting of chimeric and humanized Abs.

The logic of this development has been to reduce the size of the regions of murine origin in the variable domains of these therapeutic Abs, notably in the framework regions (humanized Abs), finally going toward fully human antibodies. The improvement of tolerance that could then be observed (notably decrease in the frequency of appearance of anti-therapeutic-Ab Abs and/or increase in lifetime of therapeutic Abs) reflects the soundness of this approach, even if the tolerance expected from the human type G immunoglobulins (IgG) is not perfect, as shown by earlier works on idiotypic networks for example (Behn U. Idiotypic Networks: Toward a Renaissance? Immunol Rev 2007; 216: 142-52, and Sfikakis P P. The First Decade of Biologic Tnf Antagonists in Clinical Practice: Lessons Learned, Unresolved Issues and Future Directions. Curr Dir Autoimmun 2010; 11: 180-210.).

The works of J. Foote's team (Hwang et al., Methods, Vol. 36, Issue 1, May 2005, pages 35-42) and of P. Thullier (Pelat et al., J Mol Biol, 2008 Dec. 31; 384(5): 1400-7) have demonstrated the possibility of “germinalizing” (“germline-humanizing”) Abs in the framework regions (FR), respectively of murine origin or derived from nonhuman macaques. According to Pelat et al., the framework regions of the Abs of nonhuman primates (NHPs) were “germinalized”, i.e. these regions were mutated to make them very similar to the sequences encoded by the human germline genes. The advantage of this approach is that the sequences encoded by the human germline genes form part of the human “immunological self”, and are therefore well tolerated during human therapeutic use. The antibody sequences encoded by the human germline genes code for the IgMs but not the IgGs, as the IgGs undergo affinity maturation, permitted owing to mutations made to the germline genes, but these mutations can be immunogenic. The mutated germline genes coding for the IgGs are called somatic genes.

The hypervariable regions (CDRs), for their part, are very exposed on the surface of the Abs, so as to interact directly with the antigens. They are therefore very likely to cause a humoral immunologic response, even more than the framework regions. However, the interaction of the Ab with the antigen is closely dependent on the sequences of the hypervariable regions, and any mutation in these regions is highly likely to alter the affinity of the Ab for its antigen.

The use of Abs in a given treatment always poses the question of immunogenicity.

There is therefore a need for Abs having good tolerance in clinical use and therefore low immunogenicity in humans, without significantly altering their affinity for the antigen. The aim of the invention is to reduce, or even cancel, the immunogenicity of the Abs during their therapeutic use, while maintaining their affinity.

The present invention relates to a strategy that aims to decrease the immunogenicity of the antibodies by mutating the somatic genes coding for the hypervariable regions of recombinant antibodies to make them closer to human germline genes coding for these regions.

The inventors recently discovered that, surprisingly, mutation of the CDR regions so as to make them very close to the sequences encoded by the human germline genes makes it possible to meet these expectations. In fact, when the hypervariable regions of Abs are mutated to make them as close as possible to the sequences encoded by the human germline genes, the problem of immunogenicity is avoided or limited since the sequences thus mutated are closer to the human “immunological self” and, surprisingly, such mutations are widely possible while preserving affinity of the Ab for its antigen comparable to the affinity of the initial Ab. Improvement of tolerance in humans is notably correlated with the decrease in immune responses directed against the antibody thus modified, owing to its greater proximity to the human “immunological self” relative to the parent antibody.

The present invention therefore relates to a method of preparing a germinalized hypervariable region of antibodies directed against a target, comprising the following steps:

a) obtaining the peptide sequence of a hypervariable region of a mammalian antibody or antibody fragment, with the exception of a human IgM, said mammal having a human homologous sequence, said antibody or antibody fragment being directed against said target;

b) comparing the peptide sequence of the hypervariable region obtained in a) with the peptide sequences encoded by the human germline genes V, (D), J, in order to identify at least one human germline gene V, (D) or J coding for the peptide sequence closest to the peptide sequence of the hypervariable region obtained in a);

c) for each different amino acid between the peptide sequence obtained in a) and the closest peptide sequence obtained in b), substitution by directed mutagenesis in vitro of each amino acid of the peptide sequence of the hypervariable region obtained in a), with the corresponding amino acid of the peptide sequence encoded by the human germline gene V, (D) or J identified in b), in order to obtain a series of mutated mammalian hypervariable regions, each peptide sequence of the hypervariable region comprising at least one mutation;

d) selecting the mutated hypervariable regions obtained in c) having an affinity for said target comparable to or greater than that of the hypervariable region of the antibody or of the antibody fragment obtained in a); and

e) optionally, preparing the peptide sequence of the hypervariable region of antibody or of antibody fragment directed against said target comprising some or all of the mutations present in at least one peptide sequence of the selection obtained in d).

Steps a) and b) can also consist of searching for the nucleotide sequences closest to the nucleotide sequences coding for the peptide sequences of the hypervariable regions of the mammalian antibody. Thus, the present invention also relates to a method of preparing a germinalized hypervariable antibody region directed against a target (hereinafter “alternative method”), comprising the following steps:

a) obtaining the nucleotide sequence of a hypervariable region of a mammalian antibody or antibody fragment, with the exception of a human IgM, said mammal having a human homologous sequence, said antibody or antibody fragment being directed against said target;

b) comparing the nucleotide sequence of the hypervariable region obtained in a) with the nucleotide sequences encoded by the human germline genes V, (D), J, in order to identify at least one human germline gene V, (D) or J coding for the nucleotide sequence closest to the nucleotide sequence of the hypervariable region obtained in a);

c) for each nucleotide that is different between the nucleotide sequence obtained in a) and the closest nucleotide sequence obtained in b), substitution by directed mutagenesis in vitro of each nucleotide of the nucleotide sequence of the hypervariable region obtained in a), with the corresponding nucleotide of the nucleotide sequence encoded by the human germline gene V, (D) or J identified in b), in order to obtain a series of nucleotide sequences of mutated mammalian hypervariable regions, each nucleotide sequence of the hypervariable region comprising at least one mutation;

d) translation of the nucleotide sequences of mutated hypervariable regions obtained in c) and selection of the mutated hypervariable regions having an affinity for said target comparable to or greater than that of the hypervariable region of the antibody or of the antibody fragment obtained in a); and

e) optionally, preparing the peptide sequence of the hypervariable region of antibody or of antibody fragment directed against said target comprising some or all of the mutations present in at least one peptide sequence of the selection obtained in d).

The method according to the invention is an in vitro method; it uses the techniques of molecular biology. This method corresponds to the germinalization of the hypervariable regions of antibodies.

To reduce the immunogenicity as far as possible, step e) preferably comprises preparing the peptide sequence of the hypervariable region of antibody or of antibody fragment directed against said target, identical to the closest sequence encoded by at least one human germline gene V, D or J identified in step b).

Moreover, in order to reduce as far as possible the immunogenicity of a variable region of antibodies, germinalization is carried out simultaneously in the framework regions and in the hypervariable regions, without explicitly distinguishing between regions of one or other type.

The present invention also relates to a germinalized hypervariable antibody region obtainable by said method. The invention also relates to an antibody or antibody fragment comprising the germinalized hypervariable region thus obtained.

Another object of the invention is the nucleic acid sequence coding for said germinalized hypervariable region or said antibody or said antibody fragment. This nucleic acid sequence can be comprised in a vector.

Finally, the present invention relates to a composition comprising said germinalized hypervariable region or said antibody or said antibody fragment.

The present invention also relates to the use of said germinalized hypervariable region or of said antibody or of said antibody fragment in therapy.

The present invention will be better understood with the aid of the following definitions.

As is well known, only one part of the antibody, the variable region, is involved in the binding of the antibody to its epitope. The constant regions of the antibody activate the immune effectors, phagocytes or killer cells, and complement; these constant regions are not involved in binding to the antigen. The antibodies comprise two identical heavy chains associated with two identical light chains, kappa or lambda. An antibody whose constant region (Fc) has been cleaved enzymatically so as to preserve its hinge region is designated as an F(ab′)2 fragment and conserves the two binding sites to the antigen. Moreover, an antibody whose constant region, including the hinge region has been cleaved enzymatically, or which was produced without this region, is designated as a Fab fragment and conserves one of the two binding sites to the antigen. The Fab fragments consist of a light chain that is bound covalently to a portion of the heavy chain called Fd.

In the variable region there are the complementarity-determining regions (CDRs), or hypervariable regions, which interact directly with the antigen. There are also regions of a second type, called framework regions (FRs, frameworks), which maintain the tertiary structure of the CDRs. These framework regions are fairly specific to the species in which the antibody was produced. In the Fd fragment of the heavy chain and in the light chain, there are four framework regions (FR1 to 4) separated respectively by three CDRs (CDR1 to 3) on each chain.

The variable region of an antibody is encoded by several genes: the variable segment (V), diversity (D) and junction (J) genes. The variable region of a heavy chain of an antibody is notably encoded by a gene V, a gene D and a gene J; the variable region of a light chain is notably encoded by a gene V and a gene J, but does not comprise a gene D. By recombination of the human germline genes V, D and J, a sequence VDJ or VJ is obtained, to which residues can be added at the junctions V/D and D/J (heavy chain) or else at the junction V/J (light chain), coding respectively for the variable parts of the heavy and light chains.

In humans, the genes of the heavy chains (locus IGH), the genes of the kappa light chains (locus IGK) and the genes of the lambda light chains (locus IGL), are respectively situated on chromosomes 14, 2 and 22. The antibodies are synthesized by recombination of the genes of the loci IGH, IGK and IGL during differentiation of the B lymphocytes.

In humans, the locus IGK comprises 76 genes IGKV (variable) of which 34 to 37 are functional and belong to 5 subgroups; the locus IGL comprises 70 to 74 genes IGLV (variable) of which 29 to 35 are functional and belong to 10 subgroups. There are five genes IGKJ (junction) situated at 3′ of the genes IGKV. Finally, the locus IGH comprises 123 to 129 genes IGHV depending on the haplotypes, of which 38 to 46 are functional and belong to 6 or 7 subgroups. Twenty-seven genes D (diversity), of which 23 are functional and nine genes JH, of which six are functional, have been described (Molecular Genetics of the Immunoglobulins, Prof. Marie-Paule LEFRANC and Gerard LEFRANC, IMGT Education).

The term “antibody” refers to an immunoglobulin molecule having the capacity to bind specifically to a particular antigen. Antibody or immunoglobulin fragments that are well known are for example the fragments F(ab′)2, F(ab)2, Fab, Fab′, Fv, scFv, diabody, dAb and Fd, or a heavy chain or a light chain, a VH or a VL.

The term “hypervariable region” refers to a CDR: 3 CDRs are present on the variable part of the heavy chain, and 3 CDRs are present on the variable part of the light chain. These hypervariable regions are in close contact with the antigen. CDR therefore means both one of the peptide sequences present on the heavy chain (i.e. CDR1, CDR2 or CDR3 of VH), and one of the peptide sequences present on the light chain (i.e. CDR1, CDR2 or CDR3 of VL). In the present application, the term “peptide sequence of a hypervariable region” means the peptide sequence of a CDR of the heavy chain or of the light chain. The 3 CDRs present on the variable part of the heavy chain and the 3 CDRs present on the variable part of the light chain form the site for attachment to the antigen. If several delimitations of the CDRs can be proposed in the context of their scientific definition, the delimitation encompassing the maximum size of each of the CDRs must preferably be adopted according to the present invention. However, any other definition is acceptable.

The mammal from which the peptide (or nucleotide) sequence of the hypervariable region of antibody or of antibody fragment used in a) is derived must have a homologous human sequence. In fact, to obtain a germinalized hypervariable region that can be administered to humans, it is important that the initial sequence has a human homologous sequence, which serves as reference for carrying out the method according to the invention. Homologous antibody of a human antibody means an antibody derived from a common ancestral sequence (common ancestor) with the human antibody.

The mammalian CDR is preferably a primate CDR. The primates can be human or nonhuman, and notably include the cercopithecoids (Cercopithecoidea) and the hominids (Hominidae). Preferably, the mammals are selected from macaque, human, chimpanzee, bonobo, gorilla, orang-utan, and baboon. Preferably, the mammals are selected from macaque and human.

The term “human germline gene” refers to any human gene present in the germ cells (spermatozoa, ova). Such a gene comprises an unmodified nucleotide sequence, i.e. that has not undergone a mutation associated with affinity maturation. The germline genes V, D and J, notably human, in the sense of the invention, have not undergone any recombination and any mutation associated with affinity maturation: they are in the germinal configuration.

Affinity maturation is based on 2 processes:
somatic mutations that affect all of the gene segments that code for the variable regions of the antibodies, i.e. the variable segments (V), the diversity segments (D) and the junction segments (J);
and selection directed by the antigen that leads to expansion of the clones which, following the mutations, express the antibodies having the highest affinity.

Thus, a human germline gene V, D or J has not undergone any somatic mutation associated with affinity maturation, and corresponds to the initial sequence present in the human germ cells. Since the invention relates to the antibodies, said human germline genes are to be understood in the present invention as those coding for the human IgMs. The human IgMs are in fact encoded in their variable part by sequences of recombinant germline genes VDJ (heavy chains) and VJ (light chains).

The invention is thus applicable to any antibody that is not encoded by human germline genes, and therefore to any antibody except the human IgMs.

“Human germline gene coding for the closest peptide sequence” to a given sequence means the human germline gene coding for the peptide sequence (or nucleotide sequence for the alternative method) that has the highest “percentage identity” with the given sequence, or the highest “percentage similarity” with the given sequence. By way of abbreviation, hereinafter in the present application, reference will be made indiscriminately to “human germline gene coding for the closest peptide sequence (or nucleotide sequence for the alternative method)” or to “closest human germline gene”.

The percentage similarity of peptide sequences takes into account the identities between amino acids, as well as the similar physicochemical properties between certain amino acids. Amino acids are said to have similar physicochemical properties if substitution of one amino acid with another does not disturb the function of the protein; this is then called conservative substitution. For example, the hydrophobic amino acid valine can be exchanged for leucine. The percentage similarity is calculated as the percentage identity, described in detail below, but takes into account the identity between amino acids and the conservative substitutions.

The percentage identity of peptide sequences is determined by comparing 2 sequences aligned optimally in a comparison window, in which the portion of amino acid sequence can comprise additions or deletions (i.e. gaps) compared with the reference sequence (which does not comprise any addition or deletion) for an optimum alignment of 2 sequences. The percentage identity can be calculated by determining the number of positions at which identical amino acid residues appear in the 2 sequences, to obtain the number of identical positions, by dividing the number of identical positions by the total number of positions in the comparison window, and by multiplying by 100 to obtain the percentage sequence identity.

Alternatively, the percentage identity can be calculated by determining the number of positions at which either the identical amino acid residue between the 2 sequences, or the amino acid residue is aligned with a gap to obtain the number of identical positions, by dividing the number of identical positions by the total number of positions in the comparison window, and multiplying by 100 to obtain the percentage sequence identity. A great many algorithms are available for aligning 2 sequences. The optimal sequence alignment can notably be found with the local homology algorithm of Smith and Waterman, 1981, Adv. Appl. Math. 2: 482, with the homology alignment algorithm of Needleman and Wunsch, 1970, J. Mol. Biol. 48: 443, with the similarity search method of Pearson and Lipman, 1988, Proc. Natl. Acad. Sci. USA 85: 2444, by computer implementations of these algorithms (GAP, BESTFIT, FASTA, and TFASTA in the GCG Wisconsin Software Package), or by visual inspection (see Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms are notably BLAST and BLAST 2.0, described in Altschul et al., 1990, J. Mol. Biol. 215: 403-410 and Altschul et al., 1977, Nucleic Acids Res. 3389-3402, respectively. A program for carrying out BLAST analyses is available from the website of the National Center for Biotechnology Information. For example, determination of sequence alignment and percentage sequence identity can use the BESTFIT or GAP program in the GCG Wisconsin Software package (Accelrys, Madison Wis.), using the default parameters.

The percentage identity of nucleotide sequences is determined as described for the peptide sequences, but this time using nucleotide sequences.

The term “germinalized hypervariable region” refers to a mammalian CDR, preferably of a primate, in which certain amino acids (or nucleotides for the alternative method) have been substituted with the amino acids (or nucleotides for the alternative method) appearing at identical position(s) in the peptide (or nucleotide for the alternative method) sequence encoded by the closest human germline gene.

The affinity K_{D} of an antibody can be measured by the conventional techniques known by a person skilled in the art. Affinity is said to be comparable between 2 antibodies for a given target when a change in affinity does not cause a functional modification on the antibody intended for therapeutic use (for example, no difference in neutralization of toxin or in cytotoxicity with respect to target cells, between the initial antibody, i.e. on which step a) is based, and the antibody, notably IgG, obtained by the method according to the invention and proposed in therapeutics). Preferably, affinities are said to be comparable between 2 antibodies for a given target when their K_{D} values vary by at most a factor of 100, preferably 50, preferably 10, preferably 5, preferably 2. In fact, in certain cases, the activity of the antibody depends little on the affinity of its binding sites to the antigen.

The affinity of an antibody A is greater than the affinity of an antibody B for one and the same target if the K_{D} of A is at least 150 times smaller, preferably at least 500 times smaller, preferably at least 1000 times smaller than the K_{D} of antibody B.

The affinity constant K_{D} can be calculated from the association and dissociation constants measured in real time by surface plasmon resonance as explained by the Biacore instrument protocols (General Electric Healthcare) or directly by competitive ELISA, for example.

The method according to the present invention therefore provides germinalized hypervariable regions. Preferably, said germinalized hypervariable region(s) is(are) present in antibody fragments of the type F(ab′)2, Fab, Fv, scFv, diabody, dAb and Fd, or in chimeric or fully human antibodies in which the Fc part of the antibody is derived from human or nonhuman homologous sequences. These antibodies or antibody fragments can comprise a single germinalized CDR, 2 germinalized CDRs, or the 3 CDRs of each of the heavy and/or light chains can be germinalized (in total, from 1 to 6 CDRs per binding site to the antigen, and from 1 to 12 CDRs per IgG, for example).

According to one embodiment of the invention, the Fc part of the antibody can be selected so as to produce IgAs, IgMs or IgGs.

According to a preferred embodiment of the invention, when the germinalized hypervariable region is present in an antibody, this antibody has a part Fc of human origin. These whole antibodies are preferred for administration in humans as they have a longer half-life than antibody fragments such as Fab, and are more suitable for intravenous, intraperitoneal, intramuscular, subcutaneous or transdermal administration. In certain embodiments of the invention, antibody fragments such as the Fab fragments are preferred for the following reasons:

i) as the Fab fragments only possess a single binding site to the antigen, immune complexes of large size cannot form,

ii) absence of an Fc region prevents the appearance of an inflammatory reaction activated by Fc, such as activation of the complement cascade,

iii) penetration of a small Fab molecule into the tissues is easier, and

iv) production of Fabs is easily performed and at low cost in bacteria such as E. coli.

Thus, according to one aspect, the method according to the present invention provides Fabs having at least one germinalized hypervariable region, or fragments of said antibody smaller or larger than Fab fragments or epitope binding peptides.

The method according to the present invention makes it possible to obtain antibodies and antibody fragments having comparable or improved affinity relative to the initial mammalian, preferably primate, antibody as well as better tolerance by the human immune system. Said “germinalized” antibody has the advantage of not inducing an immune response against itself, or inducing it less, and of having a longer half-life and of inducing fewer side effects that are harmful to the subject treated.

The method according to the invention comprises a step a) of obtaining the peptide sequence (or nucleotide sequence for the alternative method) of a hypervariable region of a mammalian, preferably primate, antibody or antibody fragment, said antibody or antibody fragment being different from a human IgM, said antibody or antibody fragment being directed against a target. As already noted, the mammal selected as starting model has a human homologous sequence; this means that to obtain a germinalized hypervariable antibody region directed against a target, the mammal selected as starting model must possess an antibody directed against said target that is a homolog of a human antibody. This step a) can be carried out according to any method known from the prior art, notably by sequencing from the native antibody or fragment thereof, or by sequencing the DNA coding for this native antibody or fragment thereof, then translation of the nucleotide sequence.

The peptide (or nucleotide) sequence of the hypervariable region is specific to a given target.

The sequence obtained in step a) is preferably a sequence of a hypervariable region of a human or macaque antibody or antibody fragment, with the exception of a human IgM.

Next, the peptide sequence of the hypervariable region obtained in a) is compared with the peptide sequences encoded by the human germline genes V, D and J. The peptide sequences encoded by the human germline genes V, D and J correspond to the peptide sequences of the variable regions of the heavy and light chains of an antibody that has not undergone affinity maturation. This comparison has the aim of identifying at least one closest human germline gene V, D or J. The aim of step b) is to identify, from among the multitude of existing human germline genes V, D and J, at least one human germline gene V, D or J having the highest percentage identity with the sequence obtained in a). In fact, the peptide sequence encoded by the human germline gene V, D or J that has the highest percentage identity with the sequence of the hypervariable region obtained in a) corresponds to the peptide sequence closest to the starting sequence. It therefore in fact determines the closest human germline gene, which is obtained at the end of step b).

Alternatively, when step a) uses nucleotide sequences (alternative method), it is also possible to compare, during said step b), the nucleotide sequences with one another, and mutate the hypervariable regions of the antibody to be germinalized (step c)) to increase the proximity with the genes V (D) J.

Preferably, step b) comprises comparing the peptide sequence of the hypervariable region obtained in a) and the peptide sequences encoded by the human germline genes V, D and J.

Preferably, step b) comprises comparison between the sequence obtained in a) and the peptide sequences encoded by all the human germline genes V, D and J, in order to identify gene V, gene J and optionally gene D (i.e. in the case of a heavy chain) coding for the peptide sequences closest to the sequence obtained in a). Owing to this step b), the human germline genes V, J and optionally D having the highest percentage identity (therefore closest) to the sequence obtained in a) are identified.

Step b) can be carried out using comparison tools online, such as VQuest or DomainGapAlign from the IMGT server (http://imgt.cines.fr); it can also be done by other means, including manually. These tools indicate the peptide sequences encoded by the human germline genes V, D and J closest to each sequence obtained in a). This analysis also makes it possible to locate the differences between the peptide sequences obtained in a) and the peptide sequences encoded by the human germline genes.

Steps a) and b) can be performed on the heavy chain and/or on the light chain of the hypervariable region.

The peptide sequence of the hypervariable region of antibody or of antibody fragment obtained in a) is then substituted, for each position at which the amino acid differs between the sequence obtained in a) and the peptide sequence encoded by the closest human germline gene V, D or J (obtained in b)), by directed mutagenesis in vitro, with the corresponding amino acid of this last sequence (step c)). For example, if a sequence of the hypervariable region of a macaque antibody is prepared in a), step c) can consist of a substitution, for each relevant amino acid, of the original amino acid of the macaque sequence with the corresponding amino acid of the peptide sequence encoded by the closest human germline gene V, D or J: we therefore have an evolution from the sequence of the macaque hypervariable region to the human germline sequence.

This step of directed mutagenesis can be performed by any appropriate method, such as by directed point mutation(s) or by gene synthesis, for example.

This step c) can be performed by mutating, one to one, the relevant amino acids, or by mutating them by group(s).

At the end of step c), we obtain a series of hypervariable regions of antibody or of antibody fragments, each of said hypervariable regions comprising one or more mutation(s). In the example in the preceding paragraph, at the end of step c), a series of hypervariable regions of macaque origin can thus be obtained, each comprising a mutation corresponding to the amino acid of the peptide sequence encoded by the closest human germline gene V, (D) or J.

Alternatively, when step a) uses nucleotide sequences (alternative method), at the end of step c), a series of nucleotide sequences of hypervariable regions of antibody or of antibody fragments is obtained, each of said hypervariable regions comprising one or more mutation(s).

Then, from the series obtained in step c), it is necessary to select the hypervariable regions of antibody or of antibody fragments having an affinity for the target comparable to or greater than the affinity of the initial hypervariable region (obtained in a)) for this same target: this constitutes step d). In fact, step d) aims to select only the mutated hypervariable regions (i.e. each comprising at least one point mutation that brings it closer to the closest human germline gene) having a comparable or higher affinity for the target, relative to the initial hypervariable region. This affinity can be measured with the Biacore apparatus or any equivalent technique as stated above.

At the end of step d), a series of mutated hypervariable regions that have approximately the same affinity or greater affinity for the target than the initial hypervariable region is thus obtained.

Alternatively, when step a) uses nucleotide sequences (alternative method), step d) comprises translation of the nucleotide sequences of mutated hypervariable regions obtained in c), then selection of the hypervariable regions of antibody or of antibody fragments having an affinity for the target comparable to or greater than the affinity of the initial hypervariable region (obtained in a)) for this same target.

Based on this selection, optionally a step e) can be added: this step e) comprises preparing a hypervariable region having some or all of the mutations present in at least one peptide sequence selected in step d). Preferably, this step e) comprises preparing a hypervariable region having all the mutations present in all the peptide sequences of the selection from step d).

The product obtained at the end of step e) thus corresponds to a synthetic sequence of mammalian antibody hypervariable region comprising one or more point mutations that bring said sequence closer to the closest human germline gene.

Thus, preferably, finally, a hypervariable region is obtained having a high identity or a high similarity with the closest human germline genes, and that has the affinity of the initial antibody (i.e. forming the basis of step a)).

Preferably, the method according to the invention also comprises, after step d) or e) (when the latter is carried out), a step f) of evaluation of the germinalized hypervariable region obtained. This step f) can comprise calculation of the percentage similarity with the sequence encoded by the closest human germline gene (or “germinality index”) for the initial peptide sequence obtained in a), and for the final peptide sequence obtained in d) or e). The germinality index according to the invention can be measured by analogy as indicated in Pelat et al., Germline Humanization of a Non-human Primate Antibody that Neutralizes the Anthrax Toxin, by in Vitro and in Silica Engineering, J. Mol. Biol. (2008) 384, 1400-1407, Pelat et al. describe the concept of germinality index for the framework regions FR only; it is possible to extrapolate this calculation similarly for the hypervariable regions, as well as for the variable regions. Briefly, the germinality index according to the present invention is calculated as follows: the closest human germline gene is translated, and the percentage of identical amino acids between the sequence translated and the peptide sequence(s) of hypervariable region obtained in a) (or d) or e)) is calculated and is called the germinality index.

Step f) can also be carried out by calculating the H-scores and G-scores. These scores are derived from the Z-score, which indicates the degree of “humanness” of a sequence: a Z-score of 0 corresponds to a sequence that is on average similar to the repertoire of human sequences, a positive Z-score corresponds to sequences that are on average highly identical to human sequences, and a negative Z-score corresponds to sequences that are on average less typical of human sequences (Abhinandan et al., Analyzing the “Degree of Humanness” of Antibody Sequences, J. Mol. Biol. (2007) 369, 852-862).

The H-score (for “humanness score”) corresponds to the Z-score calculated relative to the whole human repertoire expressed and represented in the Kabat sequence database, and the G-score (for “germline-derived score”) corresponds to the Z-score calculated relative to each part of this repertoire belonging to one and the same family, then averaged for all of these parts. The H-score and the G-score can be measured as indicated in Thullier et al., The Humanness of Macaque Antibody Sequences, J. Mol. Biol. (2010).

When the method according to the invention comprises a step f), this step can comprise calculating:
the germinality index of the peptide sequence obtained in a) and the germinality index of the peptide sequence obtained in d) or e); and/or
the H-score or the G-score of the peptide sequence obtained in a) and of the peptide sequence obtained in d) or e),

and then comparing these values with one another.

The germinality index must normally be increased between the initial peptide sequence obtained in a) and the final peptide sequence obtained in d) or e) by the method according to the invention. Moreover, the H- and G-scores must normally be increased between the initial peptide sequence obtained in a) and the final peptide sequence obtained in d) or e) by the method according to the invention.

The present invention also relates to a germinalized hypervariable antibody region obtainable by the method described above.

The invention also relates to an antibody or antibody fragment comprising the germinalized hypervariable region thus obtained. Said antibody or antibody fragment can comprise a single germinalized CDR, but can also comprise 2 or 3 CDRs of each of the heavy and/or light chains.

Said antibody or antibody fragment can also comprise, besides one or more germinalized CDRs, one or more framework regions (FR), also germinalized.

Another object of the invention is a vector comprising the nucleic acid sequence coding for said germinalized hypervariable region or said antibody or said antibody fragment.

These nucleic acids can be comprised in a recombinant vector for cloning or for expression of the antibodies of the invention.

The present invention includes all the recombinant vectors containing coding sequences for transformation, transfection or eukaryotic or prokaryotic gene therapy. It will be possible for such vectors to be prepared according to the conventional techniques of molecular biology and they will further comprise a suitable promoter, optionally a signal sequence for export or secretion, and regulatory sequences necessary for transcription of the nucleotide sequence.

A fusion polypeptide can be useful for purification of the antibodies of the present invention. The fusion domain can for example include a polyhistidine tail that permits purification on Ni+ columns, or a filamentous phage membrane anchor, which is particularly useful for database screening, according to the “phage display” technology.

One of the vectors that is suitable in the context of the invention is a recombinant DNA molecule adapted for receiving and expressing a first and a second DNA sequence, so as to permit the expression of heterodimeric antibodies such as a full-length antibody or F(ab′)2 or Fab fragments according to the invention. Such a vector provides a system for independently cloning the two DNA sequences in two separate cassettes present in the vector, so as to form two separate cistrons for expression of a first and of a second polypeptide of the heterodimeric antibody. Said expression vector is called a dicistronic vector.

The modified antibodies of the present invention can be produced in eukaryotic cells such as CHO or human or murine hybridomas for example, as well as in plant cells.

The present invention also relates to host cells, prokaryotic or eukaryotic, comprising a vector according to the invention.

Finally, the present invention relates to a composition, notably pharmaceutical, comprising said germinalized hypervariable region or said antibody or said antibody fragment.

Said pharmaceutical composition preferably comprises a pharmaceutically acceptable vehicle. Said vehicle corresponds in the sense of the invention to a nontoxic material that does not interfere with the efficacy of the biological activity of the active ingredients of the composition. The term “pharmaceutically acceptable” refers to a nontoxic material that is compatible with a biological system such as a cell, a cell culture, a tissue or an organism. The characteristics of the vehicle will depend on the method of administration.

The germinalized antibody or antibody fragment according to the invention can be labeled. Examples of markers comprise enzymes, radioisotopes, fluorescent compounds, colloidal metals, chemiluminescent compounds, and bioluminescent compounds. The methods of attaching a marker to an antibody are well known by a person skilled in the art.

Another labeling technique consists of coupling the antibody to low molecular weight haptens, and these haptens can be specifically modified by means of a second reaction. Examples of haptens are biotin, which reacts with avidin, or dinitrophenol, pyridoxal or fluorescein, which can react with specific antihapten antibodies.

The present invention relates to a method of treating a subject, preferably human, in which a therapeutically effective amount of an antibody or of an antibody fragment according to the invention is administered to said subject.

A therapeutically effective amount corresponds to an amount that is sufficient for decreasing the symptoms of the disease and the development of the infection. This amount can vary with the age and sex of the subject and the stage of the disease and will be determined by a person skilled in the art. A therapeutically effective amount can vary between 0.01 mg/kg and 50 mg/kg, preferably between 0.1 mg/kg and 20 mg/kg, and more preferably between 0.1 mg/kg and 2 mg/kg, in one or more administrations daily, over one or more days.

The method of administration can be by injection or by slow infusion. Injection can be intravenous, intraperitoneal, intramuscular, subcutaneous or transdermal.

The preparations for parenteral administration can include sterile aqueous or nonaqueous solutions, suspensions or emulsions. Examples of nonaqueous solvents are propylene glycol, polyethylene glycol, vegetable oils, such as olive oil, or injectable organic esters such as ethyl oleate. Aqueous vehicles comprise water, aqueous-alcoholic solutions, emulsions or suspensions.

The present invention will be better understood by means of the rest of the description given below, which refers to an example of obtaining an antibody according to the invention.

In the examples given below for purposes of illustration, reference will be made to the appended figures:

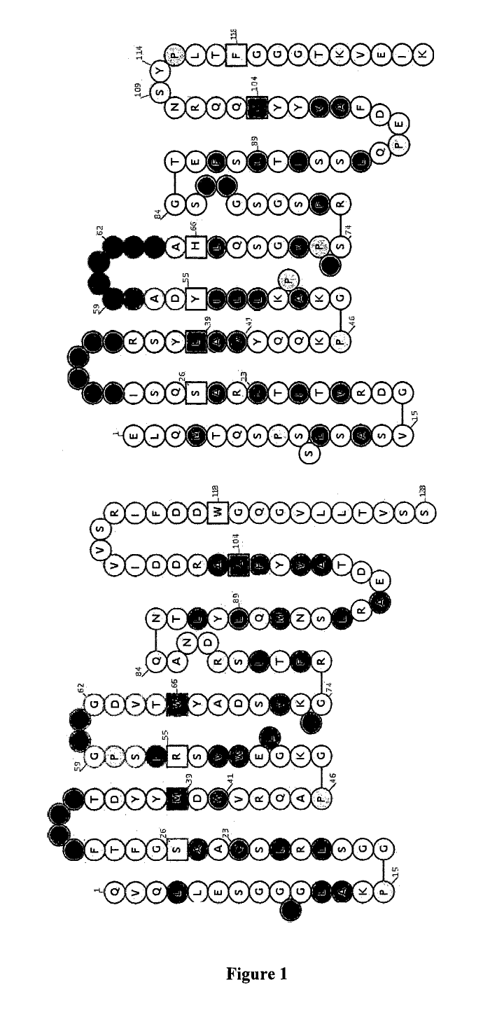

FIG. 1: string-of-pearls diagram of the variable region of the heavy chain and of the variable region of the light chain of the 45RCA antibody.

The string-of-pearls IMGT representation is effected in accordance with the IMGT numbering system.

Materials and Methods

E. coli Strains

The following E. coli strains were used:
XL1 (Stratagene, La Jolla, Calif.): recA1, endA1, gyrA96 thi-1 hsdR17 sup E44 relA1 lac [F′proAB lacIqZΔM15 Tn10(Tetr)]
SURE (Stratagene): e14(McrA) Δ(mcrCB-hsdSMR-mrr)171 endA1 supE44 thi-1 gyrA96 relA1 lac recB recJ sbcC umuC::Tn5 (Kanr) uvrC [F′ proAB lacIqZΔM15 Tn10 (Tetr)]
HB2151, used for the expression of soluble scFvs.

Antibodies and Toxins

The antibody used is an NHP scFv of very high affinity (41 pM), neutralizing ricin, called 43RCA (T. Pelat et al., BMC Biotechnology 2009, 9: 60).

The A chain of ricin, injected in the macaque, was purchased from Vector Labs, as well as complete ricin.

Antibody Selection by Phage

The particles of phages-scFv were purified and concentrated from 50 ml of culture by precipitation with PEG, then resuspended in 3 ml of PBS-BSA 1%-azide 0.02% and filtered on a 0.45 μm filter. The titer of this phage preparation was of about 5 10¹⁰ PFU/ml. The phages-scFv were submitted to three cycles of infection-selection-recovery as previously described (Andris-Widhopf, Rader et al. 2000).

Expression of Soluble scFv, Periplasmic Extraction and Purification

Each variant DNA was transformed into bacteria of the strain of E. coli called HB2151, made chemically competent. The cells were cultured at 30° C., stirred at 250 rpm in 1 L of SB medium containing 50 μg/ml of carbenicillin and 0.1% of glucose. When the culture reached an A₆₀₀ of 1.5, induction with 1 mM of IPTG was carried out for 18 h at 22° C.

The scFvs were extracted with polymixin sulfate B (Sigma) and purified on a nickel column (Ni-NTA spin column, QIAGEN, Valencia, Calif.) according to the manufacturer's instructions, then dialyzed with PBS 1× at 4° C. for 3 h.

Quantification of the scFv

The purity of the Fab was assayed by SDS-PAGE and its concentration was determined using the Quantity One® software (Biorad).

Surface Plasmon Resonance (SPR) Measurement in Real Time

The kinetic constants of the interaction between ricin and the scFvs obtained previously were determined using the Biacore X SPR system (General Electric Healthcare). The ricin was immobilized on a CM5 sensitive chip using an amine coupling procedure by injecting 30 μl of 2 μg/ml of ricin in 10 mM of sodium acetate pH 4.5. To minimize the probability of reattachment, K_{D} was measured using a high flow rate (30 μl/min) and a minimum amount of antigen coupled (about 500 RU, resonance units). The degree of binding of different concentrations of scFv in the range from 5 to 400 nM in PBS was determined at a flow rate of 30 μl/min. The binding data were introduced into a 1:1 Langmuir model of the BIA evaluation software. The association and dissociation constants (kₒₙ and k_{off} respectively) for the binding of scFv to ricin were determined at 35° C.

Sequence Analysis

The sequences of the variable regions of the heavy and light chains of the clones selected were determined by Genome Express (Meylan, France) using the primers Mkmyc and MkpelB (Kirsch et al., 2005). The sequences were analyzed online, using the IMGT system (http://imgt.cines.fr).

Results

Obtaining Antibodies Germinalized in their Framework Regions FR

The scFv 43RCA was analyzed using the DomainGapAlign tool, which indicated the human germline genes coding for sequences closest to the peptide sequences of 43RCA. These genes were IGKV1-5*01 and IGKJ4*01 for the light chain, and IGHV3-11*01 and IGHJ5*01 for the heavy chain (DomainGapAlign does not search for the genes D). The germinalization of the framework regions (or “superhumanization”) took place as shown in Table 1.

TABLE 1
Mutations corresponding to the superhumanization of 43RCA

| | | Position | No. of | | |
|---|---|---|---|---|---|
| | | (IMGT | framework | | |
| | | nomen- | region | Nature | |
| Chain | Mutation | clature) | (IMGT) | (macaque > human) | KD |
| light | 1 | 1 | 1 | E > D | 2.7 × 10−11 |
| | 2 | 2 | 1 | L > I | 2.7 × 10−11 |
| | [symbol] | [symbol] | [symbol] | [symbol] | [symbol] |
| | [symbol] | [symbol] | [symbol] | [symbol] | [symbol] |
| | 5 | 68 | 3 | Q > E | 2.3 × 10−11 |
| | 6 | 88 | 3 | S > T | 5.3 × 10−11 |
| | 7 | 97 | 3 | E > D | 5.3 × 10−11 |
| | 8 | 101 | 3 | V > T | 3 × 10−11 |
| heavy | 9 | 5 | 1 | L > V | 6.2 × 10−11 |
| | 10 | 13 | 1 | A > V | 6.2 × 10−11 |
| | 11 | 40 | 2 | D > S | 5.3 × 10−11 |
| | 12 | 42 | 2 | V > I | 3 × 10−11 |
| | [symbol] | [symbol] | [symbol] | [symbol] | [symbol] |
| | [symbol] | [symbol] | [symbol] | [symbol] | [symbol] |
| | 15 | 84 | 3 | Q > K | 2 × 10−11 |
| | 16 | 86 | 3 | T > S | 6 × 10−11 |
| | 17 | 103 | 3 | F > Y | 6 × 10−11 |
| | 18 | 122 | 4 | V > T | 4.4 × 10−11 |
| | 19 | 124 | 4 | V > L | 6.2 × 10−11 |

NB: the amino acids are designated with the single-letter code

It was therefore observed that four mutations (in position 3,4,13,14) alter the affinity; they are shown in italics and in bold in Table 1. The other fifteen mutations were combined in a synthetic gene coding for the superhumanized variant, whose affinity for ricin was measured at 2.7 10⁻¹¹ M (27 pM).

Germinalization of the hypervariable CDR regions was carried out starting from this superhumanized variant.

Obtaining Antibodies Germinalized in their Framework Regions FR and in their CDR Regions

The 15 mutations corresponding to germinalization in the hypervariable regions, and presented in Table 2, were performed (“hyperhumanization”) starting from the superhumanized variant of 43RCA.

TABLE 2
Mutations corresponding to hyperhumanization of 43RCA

| | | | No. | | Affinity |
|---|---|---|---|---|---|
| | | | of the | | of the |
| | | Position | hyper- | | variant |
| | | (IMGT | variable | | bearing |
| | | nomen- | region | Nature | the point |
| Chain | Mutation | clature) | (IMGT) | (macaque > human) | mutation |
| light | 1 | 36 | 1 | R > S | 6 × 10−11 |
| | [symbol] | [symbol] | [symbol] | [symbol] | [symbol] |
| | 3 | 65 | 2 | A > S | 6 × 10−11 |
| | [symbol] | [symbol] | [symbol] | [symbol] | [symbol] |
| | 5 | 115 | 3 | P > S | 5.4 × 10−11 |
| heavy | 6 | 35 | 1 | T > S | 6.5 × 10−11 |
| | 7 | 58 | 2 | P > S | 4.9 × 10−11 |
| | 8 | 59 | 2 | G > S | 2.5 × 10−11 |
| | 9 | 63 | 2 | D > S | 4.2 × 10−11 |
| | 10 | 64 | 2 | V > T | 6.3 × 10−11 |
| | 11 | 65 | 2 | T > I | 5 × 10−11 |
| | [symbol] | [symbol] | [symbol] | [symbol] | [symbol] |
| | [symbol] | [symbol] | [symbol] | [symbol] | [symbol] |
| | [symbol] | [symbol] | [symbol] | [symbol] | [symbol] |
| | 15 | 117 | 3 | D > Y | 8.9 × 10−11 |

It was therefore observed that five mutations (positions 2, 4, 12, 13, 14) alter the affinity; they are shown in italics and in bold. The other ten mutations were combined in a synthetic gene coding for the super- and hyperhumanized variant, whose affinity for ricin was measured at 8.9 10⁻¹¹ M (89 pM). Eleven positions out of fifteen could therefore be mutated without a significant change in reactivity of the Ab, in comparison with the affinity of the parent scFv.

The results of these works can be evaluated by measuring the Z-score (K. R. Abhinandan and A. Martin, 2007), or the germinality index (T. Pelat et al., 2008) extended to the hypervariable regions, or else parameters derived from these two.

1. A method of preparing a germinalized hypervariable antibody region directed against a target, comprising the following steps:
a) obtaining the peptide sequence of a hypervariable region of a mammalian antibody or antibody fragment, with the exception of a human IgM, said mammal having a human homologous sequence, and said antibody or antibody fragment being directed against said target;
b) comparing the peptide sequence of the hypervariable region obtained in a) with the peptide sequences encoded by the human germline genes V, (D), J, in order to identify at least one human germline gene V, (D) or J coding for the peptide sequence closest to the peptide sequence of the hypervariable region obtained in a);
c) for each amino acid different between the peptide sequence obtained in a) and the closest peptide sequence obtained in b), substitution by directed mutagenesis in vitro of each amino acid of the peptide sequence of the hypervariable region obtained in a), with the corresponding amino acid of the peptide sequence encoded by the human germline gene V, (D) or J identified in b), in order to obtain a series of mutated mammalian hypervariable regions, each peptide sequence of the hypervariable region comprising at least one mutation;
d) selecting the mutated hypervariable regions obtained in c) having an affinity for said target comparable to or greater than that of the hypervariable region of the antibody or of the antibody fragment obtained in a); and
e) optionally, preparing the peptide sequence of the hypervariable region of antibody or of antibody fragment directed against said target comprising some or all of the mutations present in at least one peptide sequence of the selection obtained in d).

2. The method as claimed in claim 1, characterized in that the mammalian hypervariable region is derived from a primate, preferably from cercopithecoids (Cercopithecoidea) or from hominids (Hominidae), preferably from macaque or from human.

3. The method as claimed in claim 1, characterized in that it comprises a step f) of calculating:
the germinality index of the peptide sequence obtained in a) and the germinality index of the peptide sequence obtained in d) or e); and/or
the H-score or the G-score of the peptide sequence obtained in a) and of the peptide sequence obtained in d) or e),
and then comparing these values with one another.

4. The method as claimed in claim 1, characterized in that the antibody fragment is selected from a heavy chain, a light chain, a VL, a VH, a Fab, a Fab′, a F(ab)2, a F(ab′)2, a scFv, a diabody, and a dAb.

5. The method as claimed in claim 1, characterized in that step b) comprises comparing the peptide sequence of the hypervariable region obtained in a) with the peptide sequences encoded by all the human germline genes V, D, J, in order to identify the human germline genes V, J and optionally D coding for the peptide sequences closest to the peptide sequence of the hypervariable region obtained in a).

6. The method as claimed in claim 1, characterized in that step e) comprises preparing the peptide sequence of the hypervariable region of antibody or of antibody fragment directed against said target comprising all the mutations present in all the peptide sequences of the selection obtained in d).

7. A germinalized hypervariable antibody region obtainable by the method as claimed in claim 1.

8. An antibody or antibody fragment comprising the germinalized hypervariable region as claimed in claim 7.

9. A nucleic acid sequence coding for the germinalized hypervariable region as claimed in claim 7.

10. A vector comprising a nucleic acid sequence as claimed in claim 9.

11. A composition comprising the germinalized hypervariable region as claimed in claim 7.

12. The germinalized hypervariable region as claimed in claim 7 for use in therapy.

13. A method of preparing a germinalized hypervariable antibody region directed against a target, comprising the following steps:
a) obtaining the nucleotide sequence of a hypervariable region of a mammalian antibody or antibody fragment, with the exception of a human IgM, said mammal having a human homologous sequence, said antibody or antibody fragment being directed against said target;
b) comparing the nucleotide sequence of the hypervariable region obtained in a) with the nucleotide sequences encoded by the human germline genes V, (D), J, in order to identify at least one human germline gene V, (D) or J coding for the nucleotide sequence closest to the nucleotide sequence of the hypervariable region obtained in a);
c) for each nucleotide that is different between the nucleotide sequence obtained in a) and the closest nucleotide sequence obtained in b), substitution by directed mutagenesis in vitro of each nucleotide of the nucleotide sequence of the hypervariable region obtained in a), with the corresponding nucleotide of the nucleotide sequence encoded by the human germline gene V, (D) or J identified in b), in order to obtain a series of nucleotide sequences of mutated mammalian hypervariable regions, each nucleotide sequence of the hypervariable region comprising at least one mutation;
d) translation of the nucleotide sequences of mutated hypervariable regions obtained in c) and selection of the mutated hypervariable regions having an affinity for said target comparable to or greater than that of the hypervariable region of the antibody or of the antibody fragment obtained in a); and
e) optionally, preparing the peptide sequence of the hypervariable region of antibody or of antibody fragment directed against said target comprising some or all of the mutations present in at least one peptide sequence of the selection obtained in d).

14. A nucleic acid sequence coding for the antibody or antibody fragment as claimed in claim 8.

15. A composition comprising the antibody or antibody fragment as claimed in claim 8.

16. A composition comprising the vector as claimed in claim 10.
